(19) United States
(12) Patent Application Publication
(10) Pub. No.: US 2001/0001660 A1
(43) Pub. Date: May 24, 2001

(54) HAIR REMOVAL COMPOSITIONI

(76) Inventors: PABLA ROMERO, GREENWICH, CT (US); OMAR ROMERO, GREENWICH, CT (US)

Correspondence Address: WILLIAM J. SAPONE, COLEMAN SUDOL SAPONE P.C., 708 THIRD AVENUE 14TH FLOOR, NEW YORK, NY, 10017, US

(21) Appl. No.: 09/444,272

(22) Filed: Nov. 19, 1999

(51) Int. Cl. A61K 7/15

(52) U.S. Cl. 424/73

(57) ABSTRACT

A hair removal composition includes bees wax, rosin and an inhibiting salt for inhibiting the irritating agents present primarily as impurities in bees wax and rosin. The composition is heated, applied to the skin and allowed to solidify, embedding hair follicles therein. After solidification, the composition is peeled off the skin with little discomfort and no substantial irritation.

### TECHNICAL FIELD

This invention relates to depilatory compositions and more particularly to a hair removal composition that removes hair with little or no discomfort or skin irritation.

### BACKGROUND

Various compositions and methods for removing hair are known. For example, hair can be removed by electrolysis, by chemical action, or by physical removal.

Physical removal generally involves the use of wax or tacky tape. The wax is typically heated above its melting point and coated onto the skin. Upon solidification, hair becomes trapped in the wax and is removed when the wax is peeled off the skin.

Various depilatory compositions which utilize physical removal action are described in, for example, U.S. Pat. Nos. 5,698,187, 5,158,765, 5,154,919, 5,840,765, 5,470,563, 4,832,949 and 2,091,313 among others.

One recognized problem with the use of waxes and rosin is that they inherently incorporate components or impurities that cause irritation that may result in redness, swelling or blisters. Several of the above referenced patents seek to address this problem by utilizing elastomers, or organo polysiloxane, or by avoiding these ingredients entirely.

In addition, such wax based systems may incorporate aggressive chemicals to soften or promote hair removal which can further cause irritation and discomfort.

What is needed is a hair removal composition which is effective in removing hair by physical action but which avoids irritation and discomfort presently encountered by the existing compositions.

### SUMMARY OF THE INVENTION

It is an object of the present invention to provide a hair removal composition which is effective in physically removing hair.

It is a further object to provide a hair removal composition which is safe and formulated with components in a specific blend which substantially avoids irritation and discomfort.

It is a further object to provide a hair removal composition that after removal, leaves the skin soft, smooth and with a refreshing feeling.

These and other objects of the present invention are achieved by a hair removal composition comprising about 8 to 25% by weight bees wax, from 70 to 90% pine rosin and from about 0.1 to 5.0% of an irritation inhibiting salt, the composition optionally formulated with a lubricant.

Utilizing these components, the aggressive irritation action of the physical impurities is tempered by the inhibiting salt which may interact with the impurities in the beeswax and pine rosin that can cause irritation. Incorporation of a lubricant, which normally would be considered to interfere with effective hair removal, instead, facilitates removal of the composition from the skin, without releasing the encapsulated hair further promoting a reduction in discomfort.

### DETAILED DESCRIPTION OF THE INVENTION

The inventive compound utilizes two key ingredients, beeswax and pine rosin. Yellow beeswax is obtained from bee honeycombs and consists of esters of straight chain monohydric alcohols with even numbered carbon chains from C₂₄ to C₃₆ esterified with straight chain acids also having even numbers of carbon atoms up to C₃₆. Propolis, pigments and unidentified substances amount to about 6% of beeswax. White beeswax is preferred, which is obtained by oxidizing yellow beeswax with peroxide or in sunlight. Beeswax has a melting point of about 62-65° C.

Pine rosin is obtained by extraction from pine wood and comprises resin acids of the abietic and primaric types, having the general formula C₁₉H₂₉ COOH and having a phenanthrene nucleus. Gum or wood rosin may be used, but preferably wood rosin having a grade of w-w (water white) is used, having a softening point of about 75° C.

The third ingredient is an inhibiting salt which interacts with the irritating agents in the bees wax and rosin to prevent irritation. This can be, for example, any carbonate compound which may be selected from the group consisting of calcium, magnesium, sodium, potassium, ammonium, etc. Preferably, calcium or magnesium carbonate or combinations thereof are used. Of course, other salts which provide a similar effect can be used.

Other optional ingredients may be used including lubricants, such as liquid petroleum jelly, honey, perfume, fillers, colorants, emollients, aloe, etc. The lubricant is a preferred optional ingredient. Present at from about 0.2 to 5% by weight, this facilitates removal of the solidified composition from the skin without causing release of the captured hair follicles.

The composition of the present invention comprises beeswax, at from about 5 to 25%, more preferably 10 to 18% and most preferably 14 to 17% and rosin at from about 70 to 90%, more preferably 80 to 90% and most preferably 85 to 90%. The inhibiting agent is present at from about 0.1 to 5%, more preferably 0.5 to 3% and most preferably at about 1 to 2%. Other optional ingredients can be added to the base composition as desired provided they do not disrupt the interaction which renders the composition non-irritant to even sensitive skin.

The composition is prepared utilizing well-known formulating techniques for mixing such ingredients. In essence, the rosin and beeswax are heated, liquefied and blended together, with the other components added thereto. Once throughly mixed, the composition is packaged in suitable containers, for either home use or professional salon use. Such use would include warming the composition to about 70-80° C., applying the composition, to a thickness of about 0.1 to 0.35″ on the skin, allowing the composition to solidify and peeling off the skin. Optionally, a cloth backing can be applied which eases removal, though this is not necessary.

### EXAMPLE

A hair removal composition having 16% bees wax, 84% rosin and 0.2% of a mix of calcium and magnesium carbonate is warmed to about 78° C. The composition is applied to the skin in long strips measuring about 12 inches long by 3 inches wide, and allowed to cool and solidify. After solidification, the composition is peeled off the skin. There is no discomfort, no redness, and no swelling or blisters. The skin is smooth, clean has a refreshing feeling and has a silky texture.

The composition has been used by individuals who have attested to the surprising lack of discomfort using the inventive composition as opposed to other waxing compositions. This is believed to be due to the interception of impurities that occur in beeswax and rosin, by the inhibiting salt, which even in near trace amounts has a beneficial effect.

After treatment, not only is the hair removed but the skin surface is smooth and has a silky texture.

Utilization of the lubricant, emollients and/or perfumes in the composition do not detract from these beneficial effects and in fact enhance the provision of what in essence is a supplemental skin treatment that leaves the skin fresh and clean. Consequently, the inventive composition is a significant advance in the art.

While preferred embodiments of the present invention has been shown and described, it will be understood by those skilled in the art that various changes or modifications can be made without varying from the scope of the invention.

What is claimed is:

1. A hair removal composition comprising beeswax at from about 8 to 25%, rosin at from about 70 to 90% and an inhibiting salt at from about 0.1 to 5% by weight.

2. The hair removal composition of claim 1 wherein the beeswax is present at from about 10 to 18%.

3. The hair removal composition of claim 1 wherein the rosin is present at from about 80 to 90%.

4. The hair removal composition of claim 1 wherein the inhibiting salt is a composition selected from the group consisting of calcium carbonate, magnesium carbonate, potassium carbonate, ammonium carbonate, sodium carbonate and combination thereof.

5. The hair removal composition of claim 1 further comprising a lubricant.

6. The hair removal composition of claim 1 further comprising an ingredient selected from the group consisting of lubricants, emollients, perfumes, colorants, honey, aloe, fillers and combinations thereof.

7. A method for removing hair from skin comprising:
providing a hair removal composition containing beeswax at from about 8 to 25% by weight, rosin at from about 70 to 90% by weight, and an inhibiting salt at about 0.1 to 5% by weight;
warming the hair removal composition to about 70-80° C.;
spreading the composition onto the skin and allowing the composition to cool and solidify such that hair follicles are trapped therein; and
peeling the composition off the skin together with the trapped hair follicles.

8. The method of claim 7 further comprising applying a strip of cloth to the spread composition, prior to solidification.
